(19) United States
(12) Patent Application Publication
(10) Pub. No.: US 2015/0088042 A1
(43) Pub. Date: Mar. 26, 2015

(54) Equine Leg Cast Rocker Attachment

(71) Applicant: Monty L. Ruetenik, Clear Lake Shores, TX (US)

(72) Inventor: Monty L. Ruetenik, Clear Lake Shores, TX (US)

(21) Appl. No.: 14/046,751

(22) Filed: Oct. 4, 2013

Related U.S. Application Data
(60) Provisional application No. 61/881,556, filed on Sep. 24, 2013.

(51) Int. Cl. A61F 5/058; A61D 99/00

(52) U.S. Cl. CPC A61F 5/0585; A61D 99/00
USPC 602/1

(57) ABSTRACT

A device or structure for fitting on the bottom of an equine leg cast to protect the cast from abrasion and wear, to aid in keeping the cast clean and, in one aspect, to provide a shaped molded rocker bottom that allows the leg cast hoof to rock forward or back, allowing the equine to find a naturally comfortable position and the method of applying the device to an equine leg cast. The device or structure consist, in general, of a solid member having a top face and a bottom face and having malleable straps attached to the solid member and extending from at least two sides of the solid member, which straps are made of material and sized to be capable of being bent or folded upward in a plane perpendicular to the top face of the solid member to attach to an equine leg cast.

### CROSS-REFERENCE TO RELATED APPLICATION

This application claims the benefit and priority from U.S. Provisional Patent Application 61/881,556 filed Sep. 24, 2013, the contents and disclosure of which is incorporated herein by reference for all purposes.

### FIELD OF THE INVENTION

A device for fitting on the bottom of an equine leg cast to protect the cast from abrasion and wear, to aid in keeping the cast clean and, in one aspect, to provide a shaped molded rocker bottom that allows the leg cast hoof to rock forward or back, allowing the equine to find a naturally comfortable position.

### BACKGROUND

Equine leg casts are used for a variety of problems such as certain bone fractures, tendon and ligament injuries, wounds, and abnormal growth and development. Increasingly, veterinarians are also casting the hoofs of laminitic horses to stabilize the hoof in order to prevent further injury. Casts are often useful as first aid tools, for immobilization of limbs, to overcome tension which helps keep skin from pulling apart at wound sites, as rigid support to allow a horse to stand and use a limb during convalescence, and for protection and reduced concussion to a limb. Casts also provide external support and reinforcement for internal fixation devices such as plates or screws used in fracture repair.

One of the more serious difficulties with a typical cast is the lack of protection on the bottom of the cast so that the fiberglass or other cast material is abraded during use and the bottom becomes soiled and otherwise damaged. Another problem is that often the casting procedure fixes the leg in an awkward position. For example, when casting horses that are lying down, the hoof may inadvertently be set in a position that is unnatural for the equine. When casting, the leg is unloaded and non-weight-bearing so there is no tension on ligaments and tendons.

It is currently customary for some practitioners to use a non-ergonomically shaped wad of casting material at the base of the cast to provide a “rocker” effect. But in so doing there is no consistency and no repeatability from cast to cast. It is important to establish better standards of care so that more practitioners can perform the same procedure without an expensive specialist.

The present invention provides a high degree of consistency so that the level of care provided to the equine can be made without the services of an expensive podiatrist or specialist or outsourcing the patient to a referral clinic. The cast attachment of this invention is cost effective and provides consistency and repeatability. The equine leg cast rocker helps to protect and stabilize the leg cast and provides more secure and natural footing for equines with a leg cast. The rocker bottom on the equine leg base allows the equine to find a comfortable position by allowing rotation or enabling a reduction of torque on the hoof.

### DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 is a bottom view of an embodiment of the invention showing the solid member with perforated metal straps.

FIG. 2 is a front edge view of an embodiment of the invention.

FIG. 3 is a rear edge view of an embodiment of the invention.

FIG. 4 is a side edge view of an embodiment of the invention.

FIG. 5 is a top perspective view of an embodiment of the invention with metal strap bent upward in position to attach to an equine leg cast.

FIG. 6 is a bottom perspective view of an embodiment of the invention with four metal straps bent upward in position to attach to an equine leg cast.

FIG. 7 is a bottom perspective view of an embodiment of the invention with three metal straps bent upward in position to attach to an equine leg cast.

FIG. 8 is a bottom perspective view of an embodiment of the invention with four fabric straps folded upward in position to attach to an equine leg cast.

FIG. 9 is a rear edge view of an embodiment of the invention.

FIG. 10 is a front edge view of an embodiment of the invention.

FIG. 11 is a side edge view of an embodiment of the invention.

FIG. 12 is a side view of a two piece solid member embodiment of the invention.

### SUMMARY

### DETAILED DESCRIPTION

In broad aspect this invention is a device and method for improving casted equine mobility and for protection of the bottom of equine leg casts. More specifically, it is, in one aspect, an equine leg cast bottom attachment comprising a solid member having a top and a bottom and having malleable or fabric straps that may be contoured to a cast extending from at least two sides of the bottom of the solid member. The straps may be bent or folded upward in a plane perpendicular to the top face of the solid member to attach to an equine leg cast.

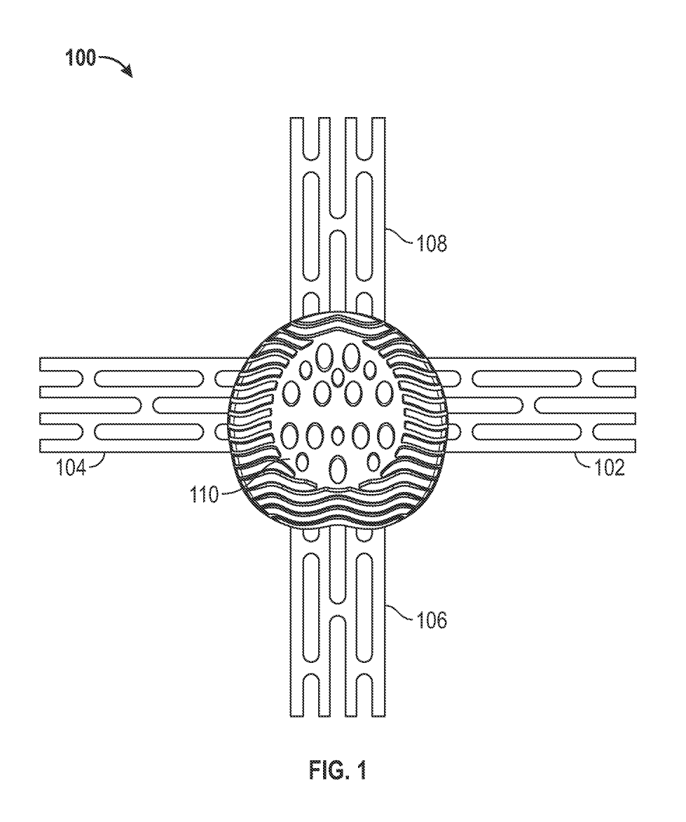

Referring to the Figures, FIG. 1 shows a bottom view of an embodiment, 100, of the invention. As shown in FIG. 1 there is a solid member, 110. The solid member has a top side and bottom side, the bottom being intended to be in contact with the ground when the device is attached to a leg cast. The bottom (110) as shown is patterned to provide more secure footing and stable contact with the ground or surface on which the equine stands. The patterning is not an essential feature of the invention but is preferred. Patterning may be any suitable design. The solid member may be of any suitable material such as wood, rubber, moldable metal and the like but molded polymer material such as polyurethane is preferred.

Metal straps 102, 104, 106 and 108 are attached to the solid member as shown. As shown the straps are fashioned into a molded polymer member about a ¼ inch from the top surface of the solid member. The straps may be attached to the solid member in any number of ways but are preferably attached near the top (in the upper half of the member) as illustrated in the figures. It is preferred that the solid member be of molded polymer material and in a preferred embodiment molded polyurethane elastomer. It is essential that the straps be sufficiently flexible to be bent or folded up and contoured into cast material to a position perpendicular to the plane of the top and bottom of the solid member in order to be attached to an equine leg cast. The straps on opposite sides may be a single piece that extends across or through the solid member and extends out on opposites sides. Thus, if the solid member is molded polymer, straps can be laid in the top of the mold, extending out from each side of the mold and the polymer poured into the mold to set, resulting in a solid member with two side straps for each strip of strap material. If the solid member is a completed piece prior to attaching the straps, for example if the solid member is wood or pre-molded polymer, the straps may be attached by any suitable conventional means, such as with screws, nails, adhesive or insertion through a slot in the solid member and similar means.

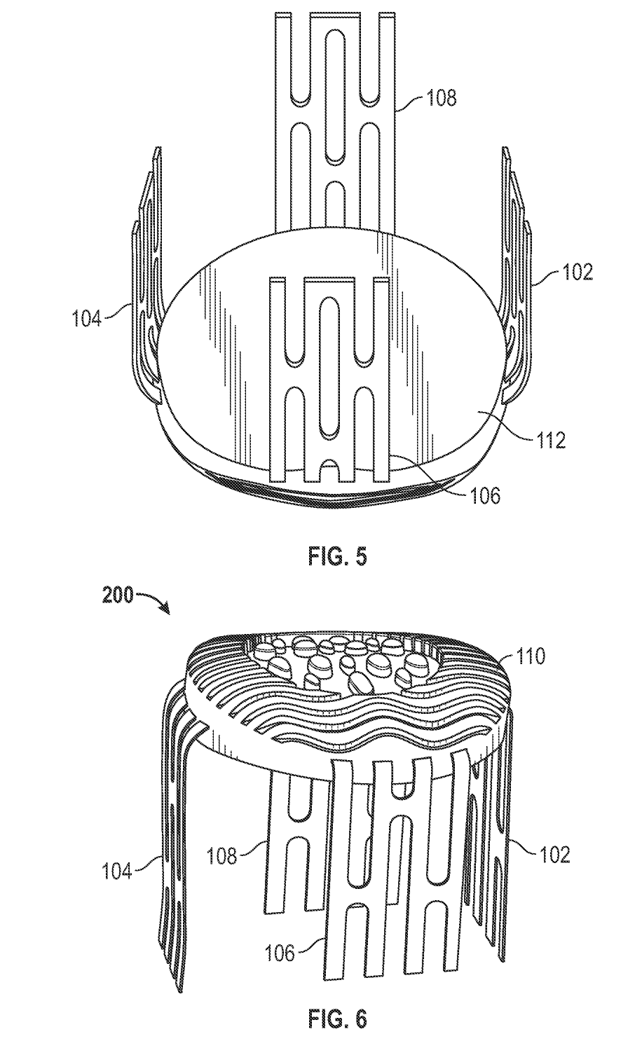

FIGS. 5, 6, 7 and 8 illustrate the straps folded up and contoured to fit a leg/cast shape—FIGS. 6, 7 and 8 show the cast support inverted from normal positioning on an equine leg cast in order to illustrate the patterned bottom. FIG. 5 shows an embodiment as it is presented in use as on a cast.

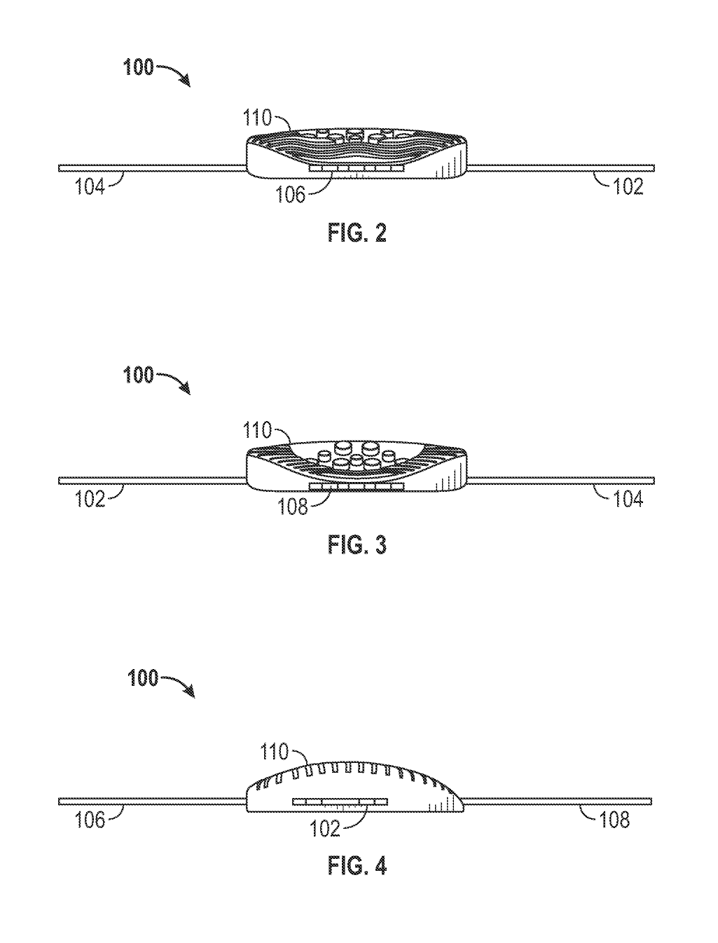

FIGS. 2, 3, 4, 9, 10, 11 and 12 are side and end views of a solid member that is tapered to form a molded rocker configuration. These Figures show the way the solid member may be tapered and the way the straps are molded in near the top side of the member. The tapers of the molded member may be varied to provide the type rocker action desired. The rocker illustrated in FIGS. 2, 3 and 4 is sloped to the front and sloped to the rear of the solid member. This allows maximum flexibility of movement and allows the horse more options to find the optimum comfortable position but is least stable of the configurations. This rocker is useful to enhance self-stretching of ligaments as when preparing for exercise, much like a person stretching the Achilles tendons by leaning on a wall before a run.

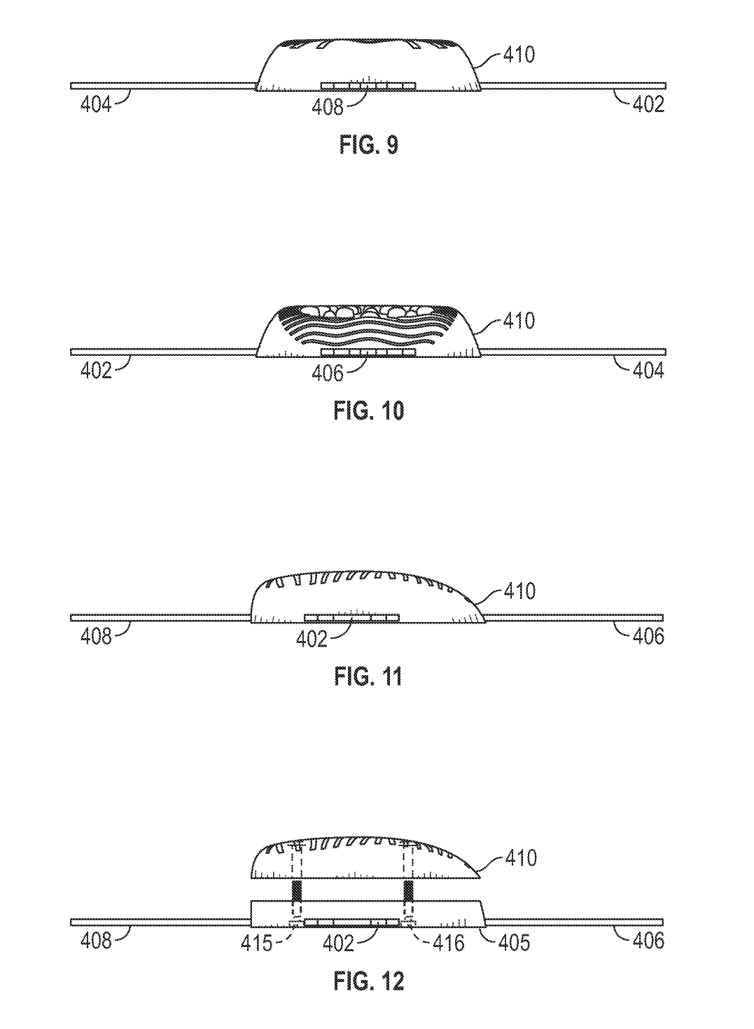

It is also suitable to have the rocker sloped only to the front, the front will be tapered as shown but the rear will be essentially flat from the center of the member to allow more stable footing as is illustrate in FIGS. 9, 10, 11 and 12. This rocker configuration provides the equine a more stable footing in a preferred neutral resting position. The neutral resting position in the rear section of the device enables the horse to have a stable place to rest, yet if there is movement fore and aft or laterally the rocker rolls to lessen torque on the tendons, ligaments and especially on compromised lamina. This configuration is generally preferred.

Both styles of rocker configuration are useful in appropriate situations and both are within the scope of this invention The narrowed toe of the rocker enhances lateral break-over and provides the equine with more flexibility of movement.

Various ways in which this solid member may be tapered are within the scope of this invention. A number of possible rocker shapes are described in co-pending US application Pub. No. 2011/0067366, Mar. 24, 2011 (Ser. No. 12/882,352), incorporated herein by reference for all purposes. The rocker shape on the bottom moves the break-over point back as well as helping isolate the lateral forces on the cast and thereby on the bone column.

An advantage of the rocker attachment is that it greatly reduces the surface area in contact with the ground or other surface onto which the equine stands. For example, a typical cast bottom would be about 8 inches×8 inches or 64 square inches. A suitable rocker attachment will be about 4 inches×5 inches or 20 square inches which is only about 30% of the surface of the cast bottom. Smaller rockers will reduce the ground contact even further.

The Figures illustrate straps of expanded or perforated metal molded into the lower portion of the rocker member that protrudes out laterally. The expanded or perforated metal reduces the weight and is more easily molded to contour to the anatomical confirmation of the horse's leg around the cast. It allows a good bond with the casting adhesive and allows the practitioner to bend it upward into the casting material.

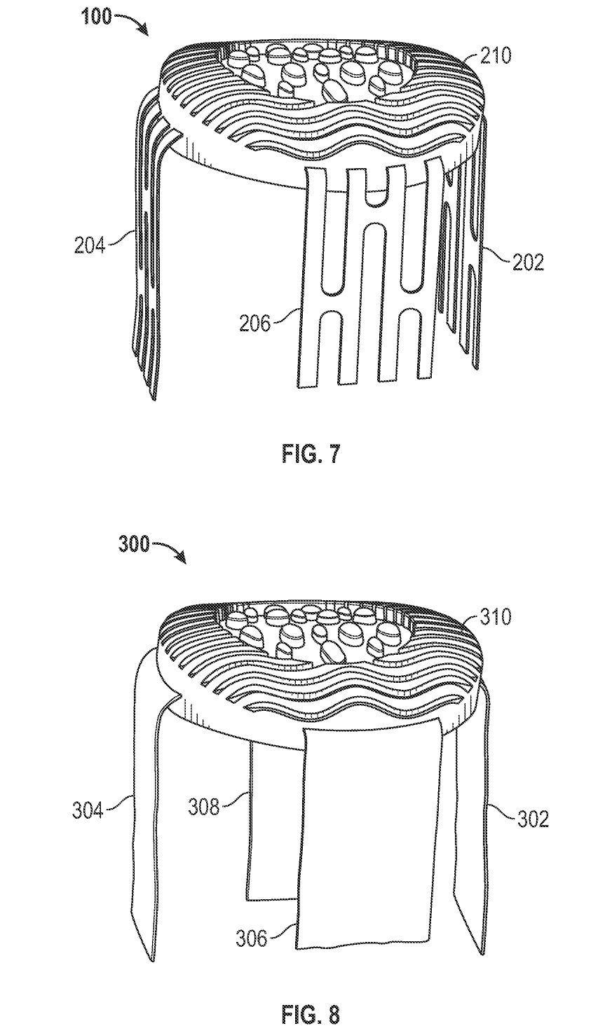

There may be only three straps extending from the sides of the solid member in one aspect of the invention as shown in FIG. 7 but four are preferred. It may also be advantageous to have five (5) straps spaced around the solid member. Five straps will position the straps in a way that will facilitate cutting the cast when it is to be removed. FIG. 1 shows the straps in the same plane as the molded member as would be suitable for packaging and shipping. But in use they are folded up and against the sides of a cast and attached to the cast material. The straps may be attached to the cast in a variety of suitable ways that will be apparent to those skilled in the art. The means of attachment will depend to some extent on the particular cast and techniques used for casting. So long as the cast protector is securely attached, it will be adequate. Since casts are usually removed with a cast cutter, the means of attachment should take into consideration any interference with removal that the attachment may cause. For example, the metal strap may be screwed to the cast, both metal and fabric straps my attached with wrapping of casting material, duct taping it to the cast, wrapped to the cast with Elastikon™ elastic medical tape and like means.

The metal straps may be of various thicknesses or composition with the criteria that they be sufficiently malleable to allow them to be formed around the cast as the cast is being applied. This malleability allows the strips to be anchored to the cast in a manner that will hold them securely in place during use. Thin strips of stainless steel or aluminum are very suitable. Relatively thin stainless has very satisfactory strength and will not rust or corrode from the casting materials as will steel. Aluminum is somewhat softer, more easily molded but generally more expensive. It is preferred that the metal straps be perforated. As shown in the Figures, straps with elongate slots about two (2) inches long and ¼ inch wide in a 1¾ width strap work very well and are illustrative of some embodiments of the invention. In this strap about forty (40)% to half the strap is cut away.

Instead of metal straps, fabrics or other flexible materials may also be used to attach the cast protector to the bottom of the cast as shown in FIG. 8. Four straps 302, 304, 306 and 308 are shown. In one aspect the fabric will be of the same material as the cast wrapping. It is generally preferred, however, to use stronger fabrics such as nylon ballistic cloth or ultra-high-molecular-weight polyethylene fabrics (Dynema™ or Spectra™). In general, it is preferred that the cloth or fabric straps be longer than the metal straps. Lengths extending from the molded bottom will be 3 to 14 inches and preferably about 6 to 12 inches. Width can vary from about one inch to about 6 inches. Wider widths can be split to provide more flexibility in wrapping them into the cast material.

Nylon ballistic cloth is generally very tough and durable fabric made with a “ballistic weave”, typically a 2×2 or 2×3 basket weave. It can be woven from nylon yarns of various denier such as 840 denier and 1680 denier.

Other particularly suitable fabrics include Dyneema and Spectra™. Dyneema and Spectra™ are Ultra-high-molecular-weight polyethylene fabrics that are gel spun through a spinneret to form oriented-strand synthetic fibers with yield strengths as high as 2.4 GPa (350,000 psi) and specific gravity as low as 0.97 (for Dyneema SK75). High-strength steels have comparable yield strengths, and low-carbon steels have yield strengths much lower (around 0.5 GPa). Since steel has a specific gravity of roughly 7.8, this gives strength-to-weight ratios for these materials in a range from 10 to 100 times higher than steel. Strength-to-weight ratios for Dyneema are about 40% higher than for aramid.

The solid member is intended to be designed and sized to provide the optimum break-over for most applications; however, when constructed of a molded elastomer it may also be customized on site of use to an individual equine or individual need with standard farrier tools such as a rasp and knives. Molded elastomer enables an attending practitioner to further customize the device for each patient after looking at radiographs or other imaging means to the position of the coffin bone relative to the ground level and watch the horse's movement with the device. The solid member is preferably made of molded elastomeric polymer. It needs to be relatively hard and rigid, but not completely so. Molded polyurethane is very suitable and convenient to work with. It is preferred that thermoplastic polyurethane of about 45 to Shore A hardness be used, with Shore A hardness of 75 to 85 being especially suitable. In some applications, softer materials—Shore A of about 50 to 55 are preferred to provide additional cushioning of the hoof. The important point is that the hardness can be easily chosen and/or adapted to the individual need of the horse to which it is applied. Polyurethanes are easily moldable in open molds or by injection molding. Other polymer materials with similar characteristics as polyurethane, such as polyvinyl chlorides, styrene butadiene styrene polymer, epoxies and the like, are also usable. Choice of these will be well within the ability of those skilled in the polymer art to select.

In another embodiment the solid member is made in two pieces—one more rigid section with the straps attached and a rocker attachment attached to the more rigid solid base. This embodiment is illustrated in FIG. 12 where part 405 is the rigid solid member and 410 the attachable rocker bottom. Straps 402, 406 and 408 are shown. As shown, the bottom, 410, is attached to the solid member base 405, by barrel screws (415 and 416), but it may also be attached in other ways as by adhesives, horse shoe nails, direct bonding and the like. A suitable solid member base in a two piece arrangement will be polyurethane of a Shore A hardness of 90 plus.

The solid member, if made of polymer, may be reduced in weight by adding low density small particles (preferably spheriodal shaped) into the polymer as is done with polymer equine boot orthotics in U.S. patent application Ser. No. 13/396,191, filed Feb. 14, 2012, and U.S. patent Ser. No. 14/046,430 file Oct. 4, 2013, the disclosures of which is incorporated herein by reference. The particless may be any material with sufficient flexibility and durability for incorporation into a shock absorbing orthotic pad. In a preferred embodiment wherein the polymer of the solid member is polyurethane, the particles materials are of lower density than elastomeric polyurethane, capable of being adhered to by elastomeric polymer materials (preferably polyurethane), and generally spherical or elliptical in shape. Suitable materials may include, but are not limited to, polymers and elastomers, and preferably expanded foam or cellular formulation of these polymers. Specific examples include polypropylene and expanded polypropylene (PP), polyethylene and expanded polyethylene (PE), high density polyethylene (HDPE), ethylene propylene diene monomer (EPDM), polystyrene (PS), polyurethane and polyurethane foams, polystyrene, polybutadiene, styrene-butadiene rubber (SBR), and polyvinyl chloride. In one embodiment, polypropylene and polyethylene are preferred, with closed-cell expanded polypropylene being particularly preferred for its low density, high durability, flexibility, resilience, and thermal insulation.

The particle cross section or diameters are desirably in the range of one (1) to six (6) millimeters (mm) (3.9×10⁻² to 2.4×10⁻¹ inches). In a preferred embodiment, the spheroids have a diameter of approximately two (2) to four (4) mm (7.9×10⁻² to 1.6×10⁻¹ inches), with approximately three (3) mm (7.9×10⁻² inches) being particularly preferred. Particles of these sizes are small enough to be incorporated into the elastomer and large enough to not unduly increase viscosity of the polymer mixture during molding. If the particles are too large the result is a kind of permanent set reducing the flexibility and compressibility of the molded piece.

One of the key properties of the particles is their low density compared to the polymer of the solid member, resulting in a lower overall weight-to-volume ratio of the member. The difference in density between the particles and the polymer causes the particles to rise towards the top of the mold during casting, which becomes the bottom of the member. Because the p particles are lower density than the polymer, they rise and accumulate at the top of the mold, which is the bottom of the pad, during molding. The member will then consist of a top layer comprised predominately of elastomer(s) that will be in contact with the hoof and a bottom layer of particle-filled polymer that provides a thermal barrier to protect the bottom of the cast, on which the device is fitted, from overheating. It is obvious that in other embodiments, the relative densities of the particles and elastomer(s) may be varied to control the relative positions of elastomer and particles.

The density of the particles is desirably in the range of about twenty (20) to five hundred twenty (520) grams/liter (g/l). For example, expanded polypropylene beads have a density range of about ten to two hundred (10-200) g/l, and preferred mid density beads have a density range of from about forty to one hundred twenty (40-120) g/l. Suitable polyurethane elastomers have densities of about one thousand twenty-five to one thousand seventy (1025-1070) g/l, so the ratio of density of elastomer to particle will be in the range of from about eight to twenty-eight (8-28). It is preferred that the particles be at least half the density of the elastomer and preferably no more than about 30% as dense.

In broad aspect, the method for manufacturing the lighter solid members comprises mixing particles with one or more elastomer component during curing to form a molded piece. In general, the basic process is to mix the elastomer components and catalysts, and to disperse the particles in unset polymer during curing while the polymer is still substantially in the liquid state. A mold of the desired size and shape is filled with the resulting mixture and the mixture is allowed to set and cure.

In a preferred embodiment, the method comprises mixing the chosen material with small particles before curing of the polymer is complete to form a homogeneous suspension. This mixture is poured into an open mold in which the open top of the mold is the top of the solid member. The particles will tend to rise to the top of the mold, accumulating in the upper portion of the molded piece. Once the elastomer is properly cured, the piece is removed from the mold. The resulting molded piece is comprised of a layer of elastomer in the upper portion of the member and a layer of elastomer-bound particles in the lower portion of the pad suitable for thermally insulating the hoof from the ground, absorbing shock, and reducing the overall weight of the member. A middle layer is comprised of an increasing concentration of particles dispersed in elastomer as one progresses from the top of the pad towards the bottom.

Casting the leg is conventional and well known by those, such as veterinarians, skilled in the art. The use of the device of this invention is not dependent on the particular kind of cast or casting material; however, in general the use of the protective device will allow use of less casting material, reducing the weight of the cast. It is customary to use fiberglass casting materials that are well suited for use with the protective device of the invention. For example, an often used method of leg casting will include first placing a fabric stocking on the leg such as a 3M™ Synthetic Cast Stockinet (a synthetic stockinet underlayment for all standard casting applications, and suitable for use with all casting materials, plaster and synthetic). This stocking may be then overlaid with casting tape such as 3M™ Vetcast™ Plus Veterinary Casting Tape 1468-4.

As the casting tape is applied, the straps of the protective device of this invention may be taped into the cast structure. Alternatively, the straps may be attached by adhesive, by screwing the straps to the finished and other such means as will be obvious to those skilled in the art.

In this specification, the invention has been described with reference to specific embodiments. It will, however, be evident that various modifications and changes can be made thereto without departing from the broader spirit and scope of the invention as set forth in the appended claims. The specification is, accordingly, to be regarded in an illustrative rather than a restrictive sense and the scope of the invention should be limited only by the appended claims.

1. An equine leg cast attachment device comprising a solid member having a top face and a bottom face and having malleable straps attached to the solid member and extending from at least two sides of the solid member, which straps are made of material and sized to be capable of being bent or folded upward in a plane perpendicular to the top face of the solid member to attach to an equine leg cast.

2. The devise of claim 1 wherein the straps are malleable metal.

3. The devise of claim 2 wherein the metal strap are perforated.

4. The devise of claim 3 wherein the perforated straps have at least 40% of the metal removed.

5. The devise of claim 1 wherein the straps are fabric.

6. The devise of claim 1 wherein the bottom of the solid member is tapered.

7. The devise of claim 6 wherein the bottom of the solid member is tapered towards the front, from the centerline of the member.

8. The devise of claim 6 wherein the bottom of the solid member is patterned on the bottom.

9. The devise of claim 1 wherein the solid member is made of a polymer.

10. The equine leg cast devise of claim 8 wherein the polymer is polyurethane.

11. The devise of claim 1 wherein the solid member comprises two pieces that are attachable together.

12. The devise of claim 1 wherein the straps extend from four sides of the solid member.

13. The devise of claim 1 wherein the elastomer solid member has incorporated therein particles of lower density to reduce the weight of the solid member.

14. A method of protecting and providing improved mobility in an equine when in a leg cast comprising fitting the devise comprising a solid member having a top face and a bottom face and having malleable straps attached to the solid member and extending from at least two sides of the solid member, which straps are made of material and sized to be capable of being bent or folded upward in a plane perpendicular to the top face of the solid member to attach to an equine leg cast on an equine leg cast by attaching straps of the device to the sides of a cast.

15. The method of claim 14 comprising fitting the devise on an equine on the bottom of a new cast on the leg, folding the straps of the device up alongside the equine leg on which some protective material has been placed and applying casting tape and casting material over the folded up straps to secure them to the finished cast.

16. The method of claim 14 wherein the solid member of the device is a polymeric elastomer, is tapered towards the front, from the centerline of the member, the straps are metal perforated straps that have at least 40% of the metal removed.

17. The method of claim 16 wherein the elastomer solid member has incorporated therein particles of lower density to reduce the weight of the solid member.

18. An equine leg cast structure comprising a solid member having a top face and a bottom face and having malleable metal straps attached to the solid member and extending from at least two sides of the solid member, which straps are made of material and sized to be capable of being bent or folded upward in a plane perpendicular to the top face of the solid member to attach to an equine leg cast, wherein the solid member of the device is a polymeric elastomer, is tapered towards the front, from the centerline of the member, the straps are metal perforated straps that have at least 40% of the metal removed.
